(19) United States
(12) Patent Application Publication
(10) Pub. No.: US 2023/0293871 A1
(43) Pub. Date: Sep. 21, 2023

(54) MICRONEEDLE BALLOON CATHETER

(71) Applicants: KING ABDULLAH UNIVERSITY OF SCIENCE AND TECHNOLOGY, Thuwal (SA); NATIONAL GUARD HEALTH AFFAIRS, Riyadh (SA); KING SAUD BIN ABDULAZIZ UNIVERSITY FOR HEALTH SCIENCES, Riyadh (SA); KING ABDULLAH INTERNATIONAL MEDICAL RESEARCH CENTER, Riyadh (SA)

(72) Inventors: Khalil MOUSSI, Thuwal (SA); Jürgen KOSEL, Thuwal (SA); Ali AHMED, Riyadh (SA); Abdelhamid SAOUDI, Riyadh (SA)

(21) Appl. No.: 18/017,978

(22) PCT Filed: Jul. 30, 2021
(86) PCT No.: PCT/IB2021/056988

§ 371 Date: Jan. 25, 2023

Related U.S. Application Data
(60) Provisional application No. 63/059,269, filed on Jul. 31, 2020.

(51) Int. Cl. A61M 37/00; A61M 25/10

(52) U.S. Cl. CPC A61M 37/0015; A61M 25/10; A61M 2025/105; A61M 2025/1081

(57) ABSTRACT

A microneedle balloon catheter includes a balloon fluidically coupled to a first fluidic channel and microneedles fluidically coupled to a second fluidic channel. The microneedles are on an exterior surface of the balloon. The catheter also includes a guiding sheath. In a first position of the microneedle balloon catheter the balloon, the microneedles, and a portion of the first and second fluidic channels are housed within the guiding sheath. In a second position of the microneedle balloon catheter the balloon and the microneedles are arranged outside of the guiding sheath. The first and second fluidic channels are fluidically isolated from each other.

### CROSS-REFERENCE TO RELATED APPLICATIONS

This application claims priority to U.S. Provisional Patent Application No. 63/059,269, filed on Jul. 31, 2020, entitled “CATHETER FOR DELIVERY AND EXTRACTION AND DELIVERY SYSTEM AND TREATMENT METHOD FOR ATHEROSCLEROSIS,” the disclosure of which is incorporated herein by reference in its entirety.

### BACKGROUND

### Technical Field

Embodiments of the disclosed subject matter generally relate to a microneedle balloon catheter for delivery of substances into the wall of a vessel of an animal or into a lumen of an organ of the animal, and more specifically to a microneedle balloon catheter that includes a plurality of microneedles.

### Discussion of the Background

Catheters are commonly used medical devices that are inserted into vessels, such as blood vessels, of animals, including humans. The design of the catheters is typically dictated by the intended medical intervention for which the catheters are to be used. For example, some balloon catheters are designed to be inserted into vessels and inflated to expand the vessel so that a stent can be placed in the vessel to allow the flow of blood through the vessels that would otherwise be impeded by, for example, the thickening of the vessel walls, such as due to plaque buildup.

US 2004/0098014 A1 discloses a catheter to both cut into an artery and deliver a drug into the artery using structures that perform both cutting and drug delivery, which are in the form of microneedles having a beveled distal portion so as to perform the cutting function to enhance dilation of an artery. Specifically, the spacing between microneedles is designed to closely approximate the effect of cutting of the artery using a continuous blade. Drug delivery can be performed using a double concentric balloon structure or the drug itself may also be the primary fluid for inflating the balloon. A double concentric balloon structure requires a complicated design and complicated production methods and using the drug to inflate the balloon can result in unnecessary spoilage of the portion of the drug that is used solely for balloon inflation. US 2004/0098014 A1 notes how medical devices used in different technological fields, such as the fields of incision-assisted angioplasty and vascular intra-mural drug delivery, are typically considered as independent applications and are not typically considered for combination into a single medical device.

In the field of endovascular drug delivery, Mercator Medsystems, Inc. has developed the Bullfrog micro-infusion catheter that uses a single hollow microneedle. The single microneedle is sheathed inside of the balloon and as the balloon is inflated, the single microneedle is uncovered and pushed into the vessel wall so that a therapeutic agent (i.e., a drug) can be injected into adventitial tissue. The single microneedle is 900 μm long, which restricts the use of the device to large vessels and it only delivers drugs into the outer layer of the vessels. Further, the single microneedle necessitates multiple injections, which requires more maneuvering of the balloon and increases the time for injecting the drug.

Thus, there is a need for a microneedle balloon catheter that is not limited in its application due to the large size of the microneedle and that does not require multiple injections to deliver the drug into a vessel wall.

### SUMMARY

According to embodiments there is a microneedle balloon catheter, which includes a balloon fluidically coupled to a first fluidic channel and a plurality of microneedles fluidically coupled to a second fluidic channel. The plurality of microneedles are on an exterior surface of the balloon. The catheter also includes a guiding sheath. In a first position of the microneedle balloon catheter the balloon, the plurality of microneedles, and a portion of the first and second fluidic channels are housed within the guiding sheath. In a second position of the microneedle balloon catheter the balloon and the plurality of microneedles are arranged outside of the guiding sheath. The first and second fluidic channels are fluidically isolated from each other.

According to embodiments there is a method of making a microneedle balloon catheter, which involves providing a balloon fluidically coupled to a first fluidic channel and a plurality of microneedles fluidically coupled to a second fluidic channel. The plurality of microneedles are affixed on an exterior surface of the balloon. The plurality of microneedles, the balloon, and a portion of the first and second fluidic channels are arranged within a guiding sheath. The first and second fluidic channels are fluidically isolated from each other.

According to embodiments there is a method of using a microneedle balloon catheter, which involves providing the microneedle balloon catheter. The microneedle balloon catheter comprises a balloon fluidically coupled to a first fluidic channel and a plurality of microneedles fluidically coupled to a second fluidic channel. The plurality of microneedles are on an exterior surface of the balloon. The catheter also includes a guiding sheath. In a first position of the microneedle balloon catheter the balloon, the plurality of microneedles, and a portion of the first and second fluidic channels are housed within the guiding sheath. In a second position of the microneedle balloon catheter the balloon and the plurality of microneedles are arranged outside of the guiding sheath. The first and second fluidic channels are fluidically isolated from each other. The method also involves inserting a portion of the guiding sheath in an animal vessel while the microneedle balloon catheter is in the first position. The microneedle balloon catheter is adjusted from the first position to the second position. While the microneedle balloon catheter is in the second position, a first fluid is provided into the first fluidic channel to inflate the balloon. Expansion of the balloon causes the plurality of microneedles to at least pierce a wall of the animal vessel. While the microneedle balloon catheter is in the second position, a second fluid is provided to the second fluidic channel so that the second fluid travels through the second fluidic channel and the plurality of microneedles into the wall of the animal vessel or into a lumen of an organ of the animal.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIGS. 1A and 1B respectively illustrate a microneedle balloon catheter in a first and second position according to embodiments;

FIGS. 10 and 1D illustrate the fluidic coupling of the microneedles balloon catheter to a fluid drive according to embodiments;

FIGS. 2A and 2B illustrate a plurality of microneedles according to embodiments;

FIGS. 3A and 3B illustrate a microneedle according to embodiments;

FIG. 4 is a flowchart of a method of making a microneedle balloon catheter according to embodiments;

FIGS. 5A and 5B illustrate a plurality of microneedles according to embodiments;

FIGS. 6A and 6B illustrate a plurality of microneedles coupled to a fluidic channel according to embodiments;

FIGS. 7A and 7B illustrate a plurality of microneedles affixed to a balloon according to embodiments

FIG. 8 is a flowchart of a method of using a microneedle balloon catheter according to embodiments; and

FIGS. 9A-9C illustrate a method of using a microneedle balloon catheter according to embodiments.

### DETAILED DESCRIPTION

The following description of the exemplary embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to the terminology and structure of catheters.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

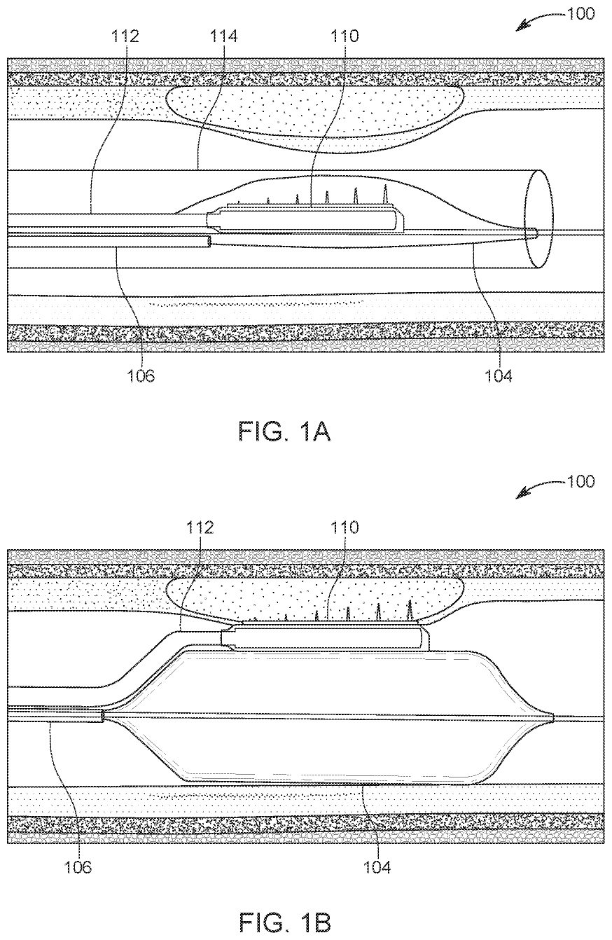

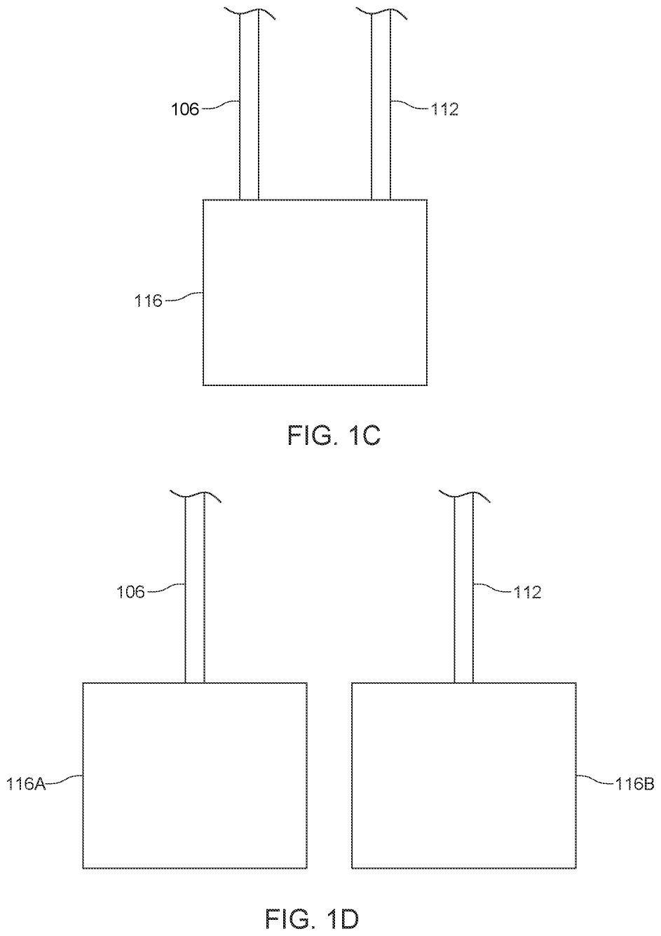

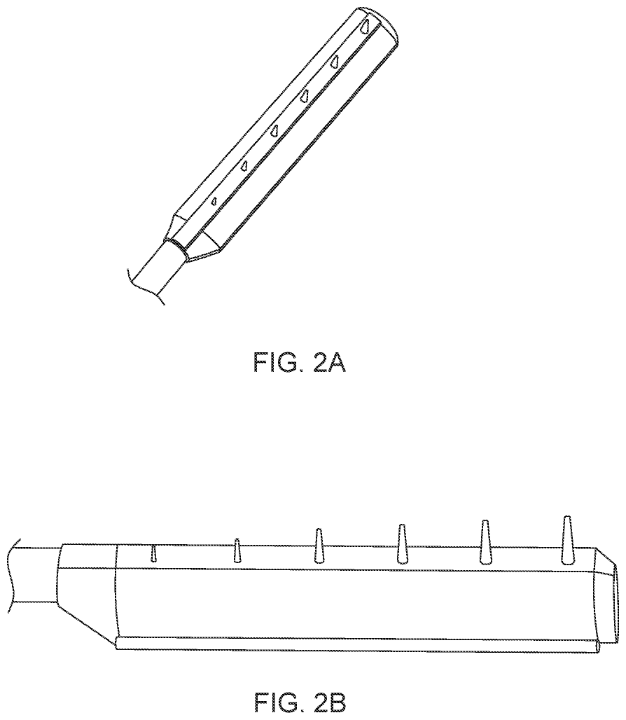

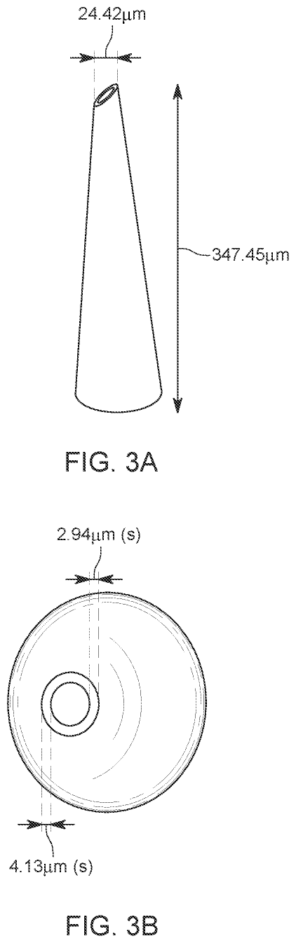

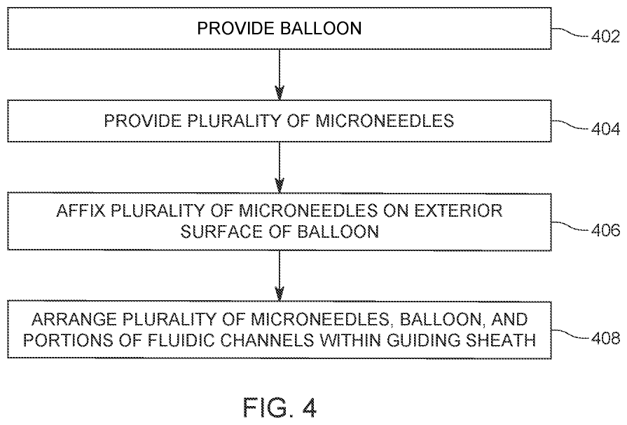

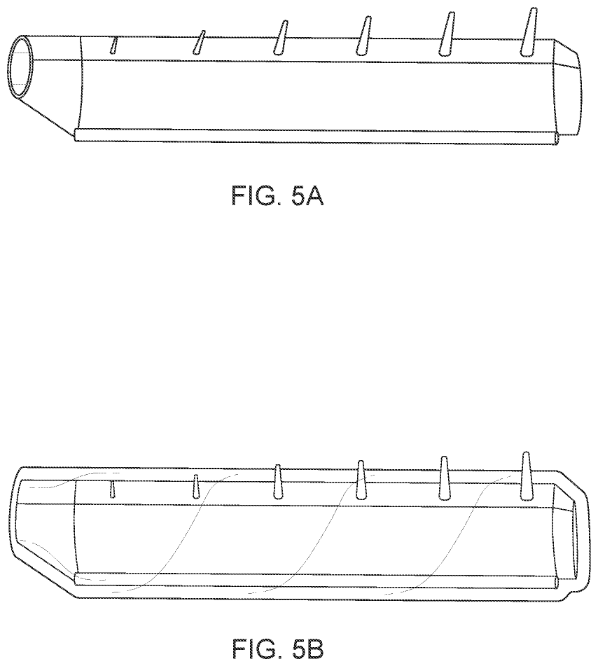

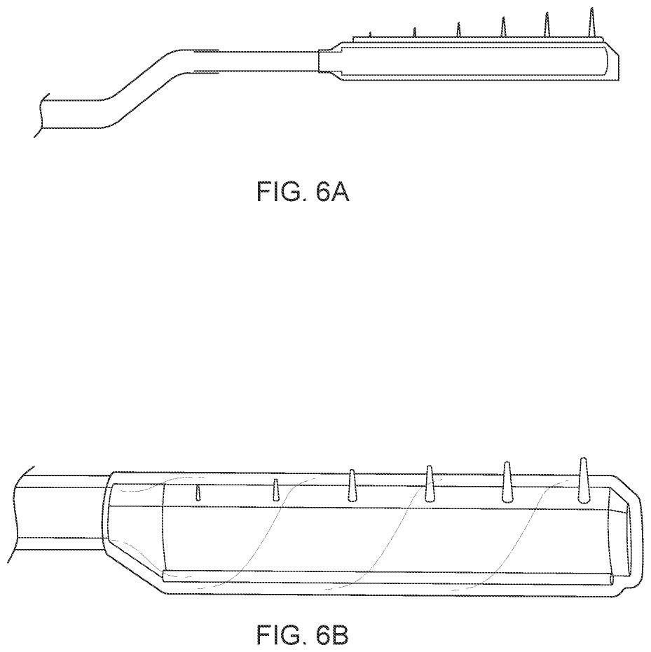

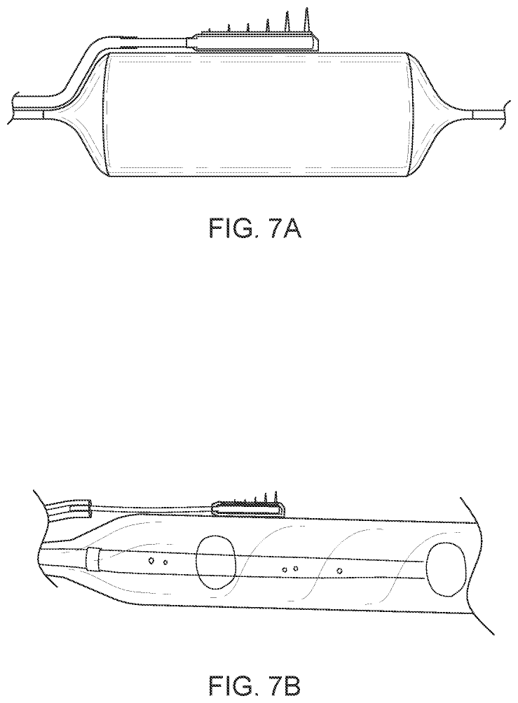

FIGS. 1A and 1B respectively illustrate a microneedle balloon catheter in a first and second position according to embodiments. The microneedle balloon catheter 100 includes a balloon 104 fluidically coupled to a first fluidic channel 106. The microneedle balloon catheter 100 also includes a plurality of microneedles 110 fluidically coupled to a second fluidic channel 112. The plurality of microneedles 110 are on an exterior surface of the balloon 104. The microneedle balloon catheter 100 further includes a guiding sheath 114. In a first position of the microneedle balloon catheter 100 (FIG. 1A) the balloon 104, the plurality of microneedles 110, and a portion of the first and second fluidic channels 106 and 112 are housed within the guiding sheath 114. Another portion of the first and second fluidic channels 106 and 112 are outside the sheath so that they can be coupled to a fluidic drive(s) as discussed below. In a second position of the microneedle balloon catheter 100 (FIG. 1B) the balloon 104 and the plurality of microneedles 110 are arranged outside of the guiding sheath 114. The first and second fluidic channels 106 and 112 are fluidically isolated from each other.

As illustrated in FIG. 1A, when the microneedle balloon catheter 100 is in the first position and balloon 104 and the plurality of microneedles 110 are inside of the guiding sheath 114, the plurality of microneedles are covered by folds of the balloon. As also illustrated in FIG. 1A, the plurality of microneedles 110 are fluidically connected with the second fluidic channel 112, which runs below the plurality of microneedles and is interposed between the plurality of microneedles and the balloon 104.

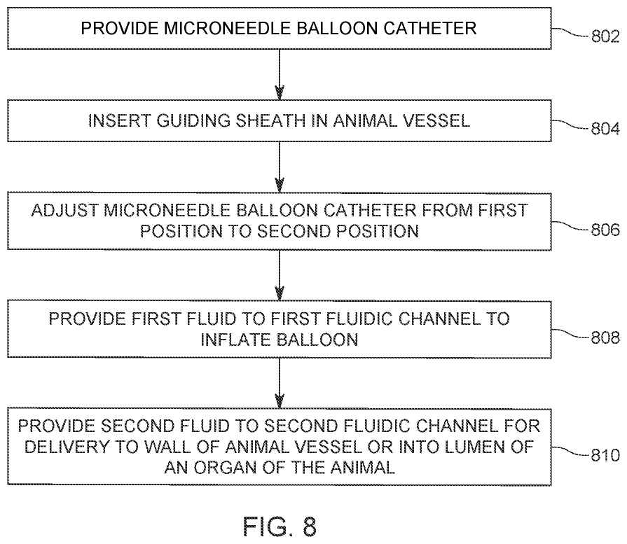

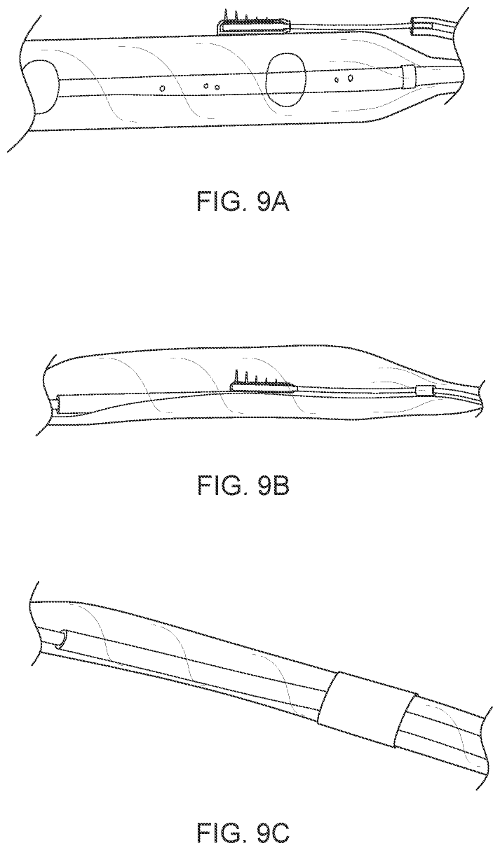

As illustrated in FIG. 10, the ends of the first and second fluidic channels 106 and 112 that are opposite of the ends directly coupled to the balloon 104 and the plurality of microneedles 110, respectively, are coupled to a fluid drive 116. The fluid drive delivers fluid into the fluidically isolated channels 106 and 112. Specifically, as described in more detail below, the fluid drive provides a first fluid through the first fluidic channel 106 to inflate the balloon 104 and the fluid drive provides a second fluid for delivery to a vessel of an animal or into a lumen of an organ of the animal via the plurality of microneedles 110. The first fluid can be any type of fluid, such as air, compressed air, an inert gas, water, saline, or any other type of inert liquid. The second fluid can be a drug or a dye. As illustrated in FIG. 1D, separate fluid drives 116A and 116B can be provided for the first and second fluidic channels 106 and 112. The fluid drive 116 or drives 116A and 116B can be manually operated pumps or can be motor-driven pumps. The first and second fluids can be stored within the fluid drive 116 or drives 116A and 116B or can be stored in an external container that it fluidically coupled to the fluid drive 116 or fluid drives 116A and 116B.

Additional details of the plurality of microneedles 110 are illustrated in FIGS. 2A, 2B, 3A, and 3B. FIGS. 2A and 2B illustrate that the plurality of microneedles 110 are arranged in a line along a length of the balloon 104. However, the plurality of microneedles can also be arranged in a two-dimensional array across the upper surface of the balloon 104. Further, the plurality of microneedles can be arranged in a plurality of one- or two-dimensional arrays on different surfaces of the balloon 104. For example, the plurality of microneedles can be arranged with several one- or two-dimensional arrays on the top, bottom, and/or side surfaces of the balloon, and further the one-dimensional array of microneedles can be aligned across the circumference of the balloon in a direction perpendicular to the insertion direction of the microneedle balloon catheter 100, i.e., perpendicular to the direction of the illustrated microneedles.

FIGS. 2A and 2B illustrate that the plurality of microneedles 110 have different dimensions, which in the illustration includes the height of the microneedles and a diameter of the base of the microneedles. As discussed below, a microneedle balloon catheter was fabricated in which the height of the microneedles increased in increments of 50 μm from 100 to 350 μm with a pitch of 600 μm, and with the inner and outer diameters of the microneedle tips being 19 and 25 μm, respectively. As illustrated in FIGS. 3A and 3B, the distal end of the microneedles are tapered and include an opening at the end to deliver fluid into the wall of an animal vessel or into a lumen of an organ of the animal. It should be recognized that although the microneedles of the plurality of microneedles illustrated in the figures have different heights, some or all of the microneedles of the plurality of microneedles can have the same height. Further, instead of being tapered, some or all of the microneedles of the plurality of microneedles can be straight, i.e., the base and tip have the same diameter.

A method for making a microneedle balloon catheter 100 will now be described in connection with FIGS. 4, 5A, 5B, 6A, 6B, 7A, and 7B. A balloon 104 fluidically coupled to a first fluidic channel 106 and a plurality of microneedles 110 fluidically coupled to a second fluidic channel 112 are provided (steps 402 and 404). As illustrated in FIGS. 5A, 5B, 6A, and 6B, the plurality of microneedles 110 can be formed first and then they are coupled to the second fluidic channel 112. However, the plurality of microneedles 110 and the second fluidic channel 112 can be formed in a single process so that they are integral. It should be recognized that the order of providing the balloon 104 and the plurality of microneedles 110 is immaterial.

One particularly advantageous technique for forming the plurality of microneedles 110 is using three-dimensional printing. For example, the plurality of microneedles can be three-dimensionally printed and can then be attached to the second fluidic channel 112. In a non-limiting embodiment, the plurality of microneedles can be made of IP-S photoresist, which provides the ability to print feature size ranging from the submicron to the millimeter scale, produces smooth surfaces, and experiences low shrinkage effects. In one embodiment, the portion of the second fluidic channel 112 closest to the plurality of microneedles can be curved with a radius conforming to an outer dimension of the balloon 104 in the inflated state, an example of which can be seen in FIGS. 6A and 7A. Additional details of a non-limiting example of producing the plurality of microneedles will be described below in connection with a microneedle balloon catheter that was produced and tested with rabbit aortic tissue.

Next, the plurality of microneedles 110 are affixed on an exterior surface of the balloon 104 (step 406). This is illustrated in FIGS. 7A and 7B but with the balloon 104 being inflated. Because the plurality of microneedles 110 are arranged above the second fluidic channel 112, the second fluidic channel 112 is interposed between the plurality of microneedles 110 and the balloon 104. The plurality of microneedles 110, the balloon 104, and a portion of the first and second fluidic channels 106 and 112 are arranged within a guiding sheath 114 (step 408). Again, the first and second fluidic channels 106 and 112 are fluidically isolated from each other.

A method of using a microneedle balloon catheter 100 will now be described in connection with FIGS. 8 and 9A-9C. Initially, a microneedle balloon catheter 100 is provided having the configuration described above in connection with FIGS. 1A and 1B (step 802). A portion of the guiding sheath 114 is inserted into in an animal vessel while the microneedle balloon catheter 100 is in the first position (step 804). Once the microneedle balloon catheter is positioned at the desired location within the animal vessel, the microneedle balloon catheter 100 is adjusted from the first position to the second position (step 806). While the microneedle balloon catheter 100 is in the second position, a first fluid is provided into the first fluidic channel 106 to inflate the balloon 104 (step 808), and the inflated balloon 104 is illustrated in FIG. 9A. Expansion of the balloon 104 causes the plurality of microneedles 110 to at least pierce a wall of the animal vessel or to push the distal portion of the plurality of microneedles into a lumen of an organ of the animal (the organ being adjacent to the animal vessel in which the balloon 104 is located). Also, while the microneedle balloon catheter 100 is in the second position and after the balloon is inflated, a second fluid is provided to the second fluidic channel 112 so that the second fluid travels through the second fluidic channel 112 and the plurality of microneedles 110 into the wall of the animal vessel or into a lumen of an organ of the animal (step 810).

After the appropriate amount fluid has been provided via the plurality of microneedles 110, the balloon 104 is deflated. As illustrated in FIG. 9B, deflating the balloon 104 causes the balloon 104 to fold on top of the plurality of microneedles 110. The deflated balloon 104, which covers the plurality of microneedles 110, is then retracted into the sheath 114. A partial retraction is illustrated in FIG. 9C.

A microneedle balloon catheter consistent with the discussion above was produced and tested on rabbit aortic tissue. As noted above, the microneedles had a height from 100 to 350 μm with an increment of 50 μm toward the proximal end of the catheter. Six microneedles were arranged along a line parallel to the catheter's axis with a pitch of 600 μm. The tips of the microneedles had inner and outer diameters of 19 and 25 μm, respectively. The structure was made of IP-S photoresist.

As discussed above, the plurality of microneedles are formed by three-dimensional printing. The plurality of microneedles of the microneedle balloon catheter that was produced and tested used the Nanoscribe Photonic Professional GT laser lithography system (Nanoscribe GmbH, Germany) for three-dimensional printing. A focused laser beam induced IP-S (Nanoscribe GmbH, Germany) polymerization at 780 nm wavelength, ≈150 mW average power, and 100 mm s−1 scan speed was performed on the three-dimensionally printed plurality of microneedles. Subsequently, the three-dimensionally printed plurality of microneedles were submerged in a fresh solution of mr-DEV 600 (Microresist Technology GmbH, Germany) for 15 min to clear unpolymerized resist, followed by an additional 15 minutes in the developing solution under vacuum. Afterward, the three-dimensionally printed plurality of microneedles was submerged in isopropanol (IPA) for 15 minutes under vacuum and dried with a nitrogen gas stream. In case of residual photoresist inside of the plurality of microneedles, an additional cleaning cycle was performed.

As also noted above, the balloon is folded around the plurality of microneedles when inside the sheath. The folding of the balloon involved deflating the balloon and pressing it from both sides to make it flat. The balloon was then folded symmetrically to the catheter axis and clamped using a mini vise. Afterward, the clamped balloon and vise were left in the oven for 1 hour at 90° C. The heating temperature and time were optimized based on different trial tests to get a V-shape folded balloon when deflated.

The assembly of the three-dimensional printed microneedles on the balloon catheter involved two steps. First, the three-dimensional printed channel (i.e., the second fluidic channel) was connected to a 30G stainless steel blunt tip needle using a Loctite 4011 instant glue, an example of which is illustrated in FIG. 6A. As also illustrated in FIGS. 6A, the other end of the needle was connected to a flexible Tygon tubing. Due to the use of Tygon tubing, which has a large wall thickness, it was not practical to attach the three-dimensional printed channel directly to the Tygon tubing was not. Direct attachment of the Tygon tubing would result in a step higher than the microneedle shaft length, which would impair the penetration. It should be recognized that direct attachment may be possible if tubing with thinner walls is employed. The 30G needle was used as a link between the Tygon tubing and 3D printed channel. Moreover, the Tygon tube allowed a reliable fluidic connection to the plurality of microneedles at a deflated and an inflated state of the balloon, as illustrated in FIGS. 7A and 7B. Second, the balloon catheter was inflated using a syringe pump to its nominal outer diameter (3 mm at 7 atm), which was confirmed with a caliper. Then, the 3D printed channel and tubing were fixed on top of the balloon catheter using a Loctite 4011 instant glue, as also illustrated in FIGS. 7A and 7B. A 5 mL syringe was connected to the flexible Tygon tube and deionized (DI) water injections were used to test for blockage and confirm effective water delivery through the plurality of microneedles.

The balloon catheter's inflation and deflation should not be altered by adding the plurality of microneedles and fluidic channel onto the balloon's surface. Furthermore, there should not be any blockage in the fluidic channel and plurality of microneedles. Consequently, a fluidic test was performed before and after assembling the three-dimensionally printed plurality of microneedles and fluidic channels on top of the balloon catheter. DI water was injected manually using a 5 mL syringe, confirming the continuous fluid flow. A second test consisted of multiple inflation/deflation cycles of the assembly set (the three-dimensionally printed microneedles channel and tubing attached to the balloon catheter). Although the system was designed for a single-use application, it was tested for 20 cycles, and no malfunction was observed.

Polydimethylsiloxane (PDMS) phantom vascular tissue experiments were performed to determine the ability to penetrate using different geometrical dimensions of the plurality of microneedles and to investigate the depth of the plurality of microneedles penetration at different balloon pressures. A customized fluidic channel with 10 microneedles was designed and affixed to a balloon catheter (balloon outer diameter was 3 mm). The first five of the plurality of microneedles had a tip diameter and wall thickness of 30 and 5 μm, respectively, while the second 5 of the plurality of microneedles had a tip diameter and wall thickness of 25 and 3 μm, respectively. The plurality of microneedles height was gradually increasing from 100 μm to 350 μm, with an increment of 50 μm toward the center. For a better X-ray contrast, the plurality of microneedles were coated with 100 nm of gold.

As the elastic modulus of a rabbit aorta ranged from 0.05 MPa to 0.5 MPa, a phantom vascular tissue with a higher modulus of elasticity was selected to test the mechanical stability of the designed microneedles. The vascular tissue model was made of PDMS with a 10:1 base to curing agent weight ratio that corresponds to an elasticity modulus of 2.61 MPa. The PDMS (Sylgard 184 Silicone Elastomer, Dow Corning Corp., MI) cylinder was fabricated by mold casting and had a length, outer diameter, and thickness of 50 mm, 3 mm, and 250 μm.

From the results, with a minimum tip diameter and wall thickness of 26 and 3 μm, respectively, the plurality of microneedles were able to penetrate a phantom vascular tissue with no mechanical failure. The 10 microneedles penetrated successfully, and different penetration depths were observed, depending on the plurality of microneedles heights. This confirmed the possibility to tailor the depth of the delivery location within the tissue. The gap between the plurality of microneedles' base in the center and the PDMS shell is presumably due to the bed of needles effect.

The final microneedle balloon catheter illustrated in FIG. 7B was used to investigate the plurality of microneedles penetration depth into the PDMS phantom vascular tissue at different balloon pressures. The penetration depth was measured for each MN from the X-ray images. The plurality of microneedles penetration depth can be controlled by selecting the microneedle height and the balloon inflation pressure. For a 250 μm thick PDMS phantom tissue, the microneedle penetration depth can be precisely controlled from about 35 μm to 240 μm by selecting the right combination of microneedle height and balloon inflation pressure.

To assess the penetration of the plurality of microneedles into the aortic tissue upon balloon inflation, a histological analysis was performed. Before the sectioning, the aortic tissue went through a fixation step in formalin, dehydration/clearing process. It was then embedded in a paraffin wax block to finally reach the sectioning step with a microtome. Even though it was attempted to align the cutting axis with the plurality of microneedles, a small deviation from it is sufficient to prevent capturing of all 6 plurality of microneedles penetration sites in one section. With a microneedle height equal to or higher than 300 μm, the plurality of microneedles can reach the adventitia of the rabbit's aortic tissue. In comparison, microneedles with heights between 250 and 150 μm reached the rabbit aorta's media layer.

Interestingly, the histological image showed no damage or rupture of the tissue, especially in the surroundings of the penetration sites. The sectioned tissue's fluorescent image showed the rhodamine B dye spreading. In particular, a higher dye concentration was observed in the penetration sites of the microneedles compared to the surrounding tissue. Based on an image analysis of the fluorescent histological section of the rabbit aorta using ImageJ software, the red dye area was about 24% in the surrounding of the plurality of microneedles with heights between 150 and 250 μm compared to about 7% for heights of 300 μm and above.

In the second test, the balloon catheter with plurality of microneedles was inflated inside a rabbit aorta, and fluorescein isothiocyanate (FITC) dye was injected into the tissue. Specifically, a fresh rabbit abdominal aorta was collected from a white male New Zealand rabbit (3-4 kg weight). The aortic tissue was washed and preserved in a saline solution for the ex vivo experiment within 12 hours after euthanasia. A syringe loaded with FITC (Sigma Aldrich, USA) dye was connected to the flexible Tygon tube, which was connected to the fluidic channel with the plurality of microneedles. The deflated balloon catheter was inserted inside the rabbit aorta and inflated. FITC was successively, manually injected into the vessel wall. The balloon catheter was then deflated and retrieved. Next, the aortic tissue was cut transversally (about 6 mm long) and opened with scissors longitudinally. Finally, the opened aortic tissue was fixed on a glass slide and imaged with a 10× objective lens on the Leica SP8 inverted confocal microscope (Leica, Germany). After opening the aortic tissue, the microneedle punctures were visible on the lumen surface, and the FITC dye injected was apparent as well.

Similar to the first test, the deflated balloon catheter was guided inside the rabbit aorta, inflated, and a fluorescent dye rhodamine B (Acros Organics, USA) was injected through the plurality of microneedles into the aortic tissue. The microneedles' penetration spots were localized with a tissue marking dye that was used as a reference for the tissue sectioning step. Then, the aortic tissue was cut transversally (about 6 mm long). The collected aorta was fixed in 10% neutral buffered formalin for 24 hours. Next, the aortic tissue was placed in a cassette and processed through a graded series of alcohols and xylenes. The tissue was then embedded in a paraffin wax block. The formalin-fixed paraffin-embedded (FFPE) sample was sectioned with a microtome at 4 μm thickness (Sakura Finetek, USA). The sectioning was parallel to the microneedle penetration line. The sections were mounted on slides and stained with hematoxylin and eosin (H&E) (Merck, Darmstadt, Germany). The cross-sectional area was imaged using an optical microscope with a 10× objective lens (Olympus BX43, Tokyo, Japan).

After each test, the delivery system was examined, and all microneedle tips (i.e., the distal ends of the microneedles) were inspected; no damage was observed, confirming the mechanical stability of the plurality of microneedles. The fluorescent dye signal was observed from the surface level of the aortic tissue to about 180 μm of depth, and it was spread out around the penetration hole, indicating the delivery inside of the tissue. The depth of the fluorescent dye signal varied linearly with the plurality of microneedles height. The penetration depth of the plurality of microneedles was affected by the high elasticity of the aortic tissue. This is a well-known effect, especially for skin penetration, where most of the microneedle displacement created a tissue indentation, and only a fraction (10-30%) of the microneedle height penetrates into the tissue. Although the 80 μm high microprobe was sharp, it deformed the internal elastic lamina and did not penetrate through. Penetration was successful with a 140 μm high sharp probe at a pressure of 67 kPa. Moreover, the balloon catheter type and size directly affected the plurality of microneedles penetration depth. Intravascular imaging techniques such as intravascular ultrasound and optical coherence tomography provide accurate measurements of the vessel size and lumen morphology. An investigation of the required balloon to vessel lumen ratio would guide the balloon size selection for a precise microneedle penetration with no vascular complications.

As discussed above, a three-dimensionally printed plurality of microneedles on a balloon catheter for endovascular drug delivery is discussed. The disclosed microneedle balloon catheter allows hollow microneedles to penetrate and deliver into the targeted area in the vessel wall or into a lumen of an organ of the animal. The results of the evaluation of the device demonstrated that the disclosed microneedle balloon catheter is capable of localized and targeted endovascular drug delivery into artery walls. The three-dimensional printing fabrication process ensures a highly customizable solution that can be tailored to the patient-specific requirements.

The disclosed embodiments provide a microneedle balloon catheter for injecting a fluid into a wall of an animal vessel or into a lumen of an organ of the animal. It should be understood that this description is not intended to limit the invention. On the contrary, the exemplary embodiments are intended to cover alternatives, modifications, and equivalents, which are included in the spirit and scope of the invention as defined by the appended claims. Further, in the detailed description of the exemplary embodiments, numerous specific details are set forth in order to provide a comprehensive understanding of the claimed invention. However, one skilled in the art would understand that various embodiments may be practiced without such specific details.

Although the features and elements of the present exemplary embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

1. A microneedle balloon catheter, comprising:
a balloon fluidically coupled to a first fluidic channel;
a plurality of microneedles fluidically coupled to a second fluidic channel, wherein the plurality of microneedles are on an exterior surface of the balloon; and
a guiding sheath, wherein in a first position of the microneedle balloon catheter the balloon, the plurality of microneedles, and a portion of the first and second fluidic channels are housed within the guiding sheath and in a second position of the microneedle balloon catheter the balloon and the plurality of microneedles are arranged outside of the guiding sheath,
wherein the first and second fluidic channels are fluidically isolated from each other.

2. The microneedle balloon catheter of claim 1, further comprising:
a fluid drive fluidically coupled to the first and second fluidic channels.

3. The microneedle balloon catheter of claim 2, wherein the fluid drive comprises a first fluid drive and a second fluid drive respectively coupled to the first and second fluidic channels.

4. The microneedle balloon catheter of claim 2, wherein the fluid drive is coupled to provide a first fluid to the first fluidic channel and a second fluid to the second fluidic channel, wherein the first and second fluids are different fluids.

5. The microneedle balloon catheter of claim 1, wherein the fluid drive is a manually operated fluid drive.

6. The microneedle balloon catheter of claim 1, wherein the plurality of microneedles are arranged in a line along a length of the balloon.

7. The microneedle balloon catheter of claim 1, wherein the plurality of microneedles are arranged in a two-dimensional array.

8. The microneedle balloon catheter of claim 1, wherein at least some of the plurality of microneedles have different dimensions.

9. The microneedle balloon catheter of claim 1, wherein a distal end of the plurality of microneedles is tapered.

10. The microneedle balloon catheter of claim 1, wherein in the first position the balloon is folded, and the plurality of microneedles are arranged within a fold of the balloon.

11. A method of making a microneedle balloon catheter, the method comprising:
providing a balloon fluidically coupled to a first fluidic channel;
providing a plurality of microneedles fluidically coupled to a second fluidic channel;
affixing the plurality of microneedles on an exterior surface of the balloon; and
arranging the plurality of microneedles, the balloon, and a portion of the first and second fluidic channels within a guiding sheath,
wherein the first and second fluidic channels are fluidically isolated from each other.

12. The method of claim 11, wherein the plurality of microneedles and the second fluidic channel are separately formed and the second fluidic channel is fluidically coupled to the plurality of microneedles.

13. The method of claim 11, further comprising:
folding the balloon around the plurality of microneedles before the plurality of microneedles, and a portion of the first and second fluidic channels are arranged within the guiding sheath.

14. The method of claim 11, wherein providing the substance delivery element comprises forming the plurality of microneedles in a line.

15. The method of claim 11, wherein providing the substance delivery element comprises forming the plurality of microneedles in a two-dimensional array.

16. A method of using a microneedle balloon catheter, the method comprising:
providing the microneedle balloon catheter, which comprises
a balloon fluidically coupled to a first fluidic channel;
a plurality of microneedles fluidically coupled to a second fluidic channel, wherein the plurality of microneedles are on an exterior surface of the balloon; and
a guiding sheath, wherein in a first position of the microneedle balloon catheter the balloon, the plurality of microneedles, and a portion of the first and second fluidic channels are housed within the guiding sheath and in a second position of the microneedle balloon catheter the balloon and the plurality of microneedles are arranged outside of the guiding sheath, wherein the first and second fluidic channels are fluidically isolated from each other;
inserting a portion of the guiding sheath in an animal vessel while the microneedle balloon catheter is in the first position;
adjusting the microneedle balloon catheter from the first position to the second position;
providing, while the microneedle balloon catheter is in the second position, a first fluid into the first fluidic channel to inflate the balloon, wherein expansion of the balloon causes the plurality of microneedles to at least pierce a wall of the animal vessel; and
providing, while the microneedle balloon catheter is in the second position, a second fluid to the second fluidic channel so that the second fluid travels through the second fluidic channel and the plurality of microneedles into the wall of the animal vessel or into a lumen of an organ of the animal.

17. The method of claim 16, wherein the first and second fluids are provided into the first and second fluidic channels, respectively, via a manually operated fluid drive.

18. The method of claim 16, wherein after the second fluid is delivered into the wall of the animal vessel or into the lumen of an organ of the animal, the method further comprising:
deflating the balloon under negative pressure;
adjusting the microneedle balloon catheter from the second position to the first position; and
removing the microneedle balloon catheter from the animal vessel.

19. The method of claim 16, wherein the first fluid is air and the second fluid is a drug.

20. The method of claim 16, wherein the first fluid is a liquid and the second fluid is a drug.
